(19) United States
(12) Patent Application Publication
(10) Pub. No.: US 2026/0053790 A1
(43) Pub. Date: Feb. 26, 2026

(54) TREATMENTS FOR CONDITIONS INVOLVING INCREASED IL-6 LEVELS

(71) Applicant: X4 Pharmaceuticals, Inc., Boston, MA (US)

(72) Inventors: Chi Nguyen, Vienna (AT); Art Taveras, Boston, MA (US)

(21) Appl. No.: 18/873,655

(22) PCT Filed: Jun. 9, 2023
(86) PCT No.: PCT/US2023/068230

§ 371 Date: Dec. 10, 2024

Related U.S. Application Data
(60) Provisional application No. 63/366,228, filed on Jun. 10, 2022.

(51) Int. Cl. A61K 31/4709; A61K 39/395; A61P 37/06

(52) U.S. Cl. CPC A61K 31/4709; A61K 39/3955; A61P 37/06

(57) ABSTRACT

Various inflammatory conditions result in increased Interleukin-6 (IL-6) levels in the human body. The present invention relates to methods of treating a variety of inflammatory conditions that results in increased Interleukin-6 (IL-6) levels in the human body with a CXCR4 inhibitor as a monotherapy or in combination with an IL-6 inhibitor.

### CROSS-REFERENCE TO RELATED APPLICATIONS

The present invention claims the benefit of U.S. Provisional Application No. 63/366,228, filed on Jun. 10, 2022; the entirety of which is hereby incorporated by reference.

### FIELD OF THE INVENTION

Various inflammatory conditions result in increased Interleukin-6 (IL-6) levels in the human body. New methods of treating these conditions with a CXCR4 inhibitor are disclosed herein.

### BACKGROUND OF THE INVENTION

Interleukin 6 (IL-6) is promptly and transiently produced in response to infections and tissue injuries, and it contributes to host defense through the stimulation of acute phase responses, hematopoiesis, and immune reactions. Although its expression is strictly controlled by transcriptional and posttranscriptional mechanisms, dysregulated continual synthesis of IL-6 plays a pathological effect on chronic inflammation and autoimmunity. For this reason, tocilizumab, a humanized anti-IL-6 receptor antibody was developed. Various clinical trials have since shown the efficacy of tocilizumab, which resulted in its approval for the treatment of rheumatoid arthritis and juvenile idiopathic arthritis. Moreover, tocilizumab is expected to be effective for other intractable immune-mediated diseases. Cold Spring Harb Perspect Biol. 2014 October; 6(10): a016295; see ncbi.nlm.nih.gov/pmc/articles/PMC4176007/.

Tocilizumab is a humanized anti-IL-6 receptor antibody, which must be administered via infusion over a 60-minute period. Tocilizumab should not be administered as an intravenous push or bolus. Tocilizumab should not be infused concomitantly in the same intravenous line with other drugs. See accessdata.fda.gov/drugsatfda_docs/label/2017/125276s1141bl.pdf.

In contrast, small molecule therapeutics such as mavorixafor may be taken orally, resulting in increased accessibility and simplicity of treatment regimes.

Thus, there is a significant, unmet need for effective small molecule therapeutics to address inflammatory conditions that involve dysregulated synthesis of IL-6, such as arthritis. The present invention addresses this need and provides certain other related advantages.

### SUMMARY OF THE INVENTION

It has now been found that certain CXCR4 inhibitors, such as mavorixafor (X4P-001), are useful in treating a variety of inflammatory conditions that results in increased Interleukin-6 (IL-6) levels in the human body, such as those described herein.

In one aspect, the present invention provides a method of treating an inflammatory condition that results in increased Interleukin-6 (IL-6) levels in the human body in a patient in need thereof, comprising administering to the patient an effective amount of mavorixafor, or a pharmaceutically acceptable salt thereof.

In another aspect, the present invention provides a method of treating an inflammatory condition that results in increased Interleukin-6 (IL-6) levels in the human body in a patient in need thereof, comprising administering to the patient an effective amount of mavorixafor, or a pharmaceutically acceptable salt thereof, in combination with an IL-6R inhibitor. In some embodiments, a dual therapy combining mavorixafor with an IL-6R inhibitor requires a lesser amount of IL-6R antibody to obtain therapeutic benefit than the corresponding IL-6R monotherapy.

### BRIEF DESCRIPTION OF THE FIGURES

FIG. 1: Co-culture of BMSC with WM cells Induces IL-6 Hypersecretion in BMSC and CXCR4 Overexpression in WM Cells.

FIG. 2: Mavorixafor Blocks BM Stroma-Induced IgM Hypersecretion in WM Cells.

FIG. 3: Even in the Absence of BMSC, Soluble IL-6 Upregulates CXCR4 Expression & IgM Secretion in WM Cells; which is Prevented by Blockade of IL-6/IL6R/STAT3 Axis. BP-1-102 is a STAT3 inhibitor.

### DETAILED DESCRIPTION OF CERTAIN EMBODIMENTS OF THE INVENTION

Interleukin-6 (IL-6) is a cytokine that has pleiotropic roles throughout the body but is most frequently associated with inflammation. IL-6 can bind to the membrane bound or soluble IL-6 receptors (IL-6R) to activate the classical or trans signaling cascades, respectively. The IL-6R lacks intrinsic signaling capacity, and the activated IL-6/6R complex must bind to gp130 to initiate downstream signaling cascades, including the JAK/STAT, ERK, and P13K/AKT pathways. The membrane bound IL-6R is only present on hepatocytes, monocytes, neutrophils, and leukocytes. In classical signaling, IL-6 binds to membrane bound IL-6R, which then binds to the ubiquitously expressed signal transducing membrane protein gp130, leading to its dimerization and activation. The classical pathway is associated with regenerative and protective effects. Meanwhile, in the trans pathway, circulating soluble IL-6R binds to IL-6 to form a complex that binds to gp130, and initiates pro-inflammatory signaling cascades.

IL-6 is traditionally considered to be a regulator of acute phase inflammatory responses and a lymphocyte stimulatory factor. During an inflammatory episode, IL-6 is highly expressed, and circulating levels of this cytokine can rise dramatically from 1-5 pg/ml to several pg/ml in certain extreme cases. Consequently, IL-6 is one of the most highly expressed mediators of inflammation. Similar increases during inflammatory episodes are not characteristic of other gp130-related cytokines. Interest in IL-6 was sparked in 2006, when IL-6 in combination with TGF-β, was shown to promote the differentiation of IL-17-secreting T helper (Th17) cells. Intervention in rheumatoid arthritis by the IL-6R monoclonal antibody tocilizumab leads to a rapid and sustained improvement in disease activity, a reduction in radiographic joint damage, and inhibition of B cell hyperactivity. These changes are also associated with a dramatic normalization of the acute phase response (including C-reactive protein [CRP]) and improvements in both pain and fatigue. See Jones et al. (2011) J Clin. Invest. 121:3375-3383, hereby incorporated by reference.

### Connection Between CXCR4-CXCL12-IL6

Detailed investigations into the role of chemokines, including stromal cell-derived factor 1a (“SDF-1α”) also known as CXCL12, and its receptor CXCR4 have now been conducted.

CXCR4 is a key regulator of normal hematopoiesis and, in particular, plays a major role in leukocyte expansion, differentiation and mobilization. CXCR4 overexpression is associated with tumor survival, growth, proliferation and metastasis. Upregulated or mutated CXCR4 promotes tight and prolonged adhesion of cancerous B-cells, or malignant lymphoplasmacytic cells, to bone marrow stroma, causing elevated expression of IL-6, IgM, CXCR4, and resistance to anti-cancer agents. Thus, the inventors have previously demonstrated that inhibition of CXCR4 was effective in treatment of cancerous conditions of B-cells, including various lymphomas, leukemias, Waldenström's Macroglobulinemia (WM), and other B-cell related disorders.

In such B-cell cancers, CXCR4 overexpression is associated with tumor survival, growth, proliferation, and metastasis. Upregulated or mutated CXCR4 promotes tight and prolonged adhesion of cancerous B-cells, or malignant lymphoplasmacytic cells, to bone marrow stroma, causing elevated expression of IL-6, IgM, CXCR4, and resistance to anti-cancer agents. While cancerous B-cells adhere to the bone marrow, they are sheltered from treatment and that excess CXC4 expression is involved in adhesion to bone marrow. The adhesion of malignant lymphoplasmacytic cells to the bone marrow results in overexpression of CXCR4 and IgM and causes hypersecretion of IL-6. Soluble IL-6 in turn upregulates CXCR4 expression and IgM secretion in cancerous B-cells. Without wishing to be bound by theory, it is believed that, by binding to and inhibiting CXCR4, mavorixafor and other CXCR4 inhibitors mobilize the cancerous B cells, to increase susceptibility to treatment. Mavorixafor thereby reduces resistance and enhances efficacy of standard of care therapeutics (chemosensitization) by selectively antagonizing the CXCR4/CXCL12/IL-6 axis with the reduction of available CXCR4 for binding by SDF-1α.

Without wishing to be restricted to any particular theory, it is hypothesized that, because CXCR4 overexpression is mechanistically-related to L-6 hypersecretion via the CXCR4/CXCL12/IL-6 axis, CXCR4 inhibitors such as mavorixafor are effective for treatment and prevention of conditions characterized by overexpression of IL-6, including rheumatoid arthritis and related disorders, and cytokine release syndrome (CRS) which may be induced by treatment with chimeric antigen receptors in recombinant T cell therapies such as tisagenlecleucel Tisagenlecleucel (KYMRIAH®, Novartis) or axicabtagene ciloleucel (YESCARTA®, Kite/Gilead).

The present disclosure demonstrates that CXCR4 inhibition not only reduces CXCR4 and IgM levels, but, unexpectedly, CXCR4 inhibition using mavorixafor as a single agent significantly reduces overexpression and hypersecretion of IL-6. It is also believed that CXCR4 inhibitors such as mavorixafor can significantly enhance the effect of IL-6 inhibitors, such as the antagonistic IL-6R antibody tocilizumab (ACTEMRA® Chugai Seiyaku Kabushiki Kaisha Corp./Roche, Genentech). Accordingly, the present invention provides materials and methods for improved treatment of conditions that involve overexpression and/or hypersecretion of IL-6.

X4P-001 (mavorixafor) is an orally bioavailable, small molecule inhibitor of CXCR4. It has now been found that CXCR4 inhibitors such as mavorixafor (X4P-001), or a pharmaceutically acceptable salt thereof or pharmaceutical composition thereof, as described in greater detail below, are useful alone for the conditions described above, and are useful as a combination therapy with one or more other therapeutic agents described herein.

In some embodiments, the CXCR4 inhibitor is selected from mavorixafor, AMD12118, and Plerixafor (AMD-3100; Sanofi).

In some embodiments, the CXCR4 inhibitor is selected from the CXCR4 inhibitors disclosed in WO2017/223229 (including I-1 through I-184); WO2017/223239 (including I-1 through I-229); WO2017/223243 (including I-1 through I-149); WO2019/126106 (including I-1 through I-69); WO2020/264292 (including I-1 through I-31); and WO2021/263203 (including I-1 through I-118); each of which is hereby incorporated by reference.

In some embodiments, the CXCR4 inhibitor is selected from the CXCR4 inhibitors disclosed in U.S. Pat. Nos. 7,291,631; 7,332,605; 7,354,932; 7,354,934; 7,501,518; 7,550,484; 7,723,525; 7,863,293; 8,778,967; 10,322,111; 7,414,065; 7,022,717; 7,084,155; 7,807,694; 6,750,348; 7,169,750; 7,491,735; and 7,790,747; each of which is hereby incorporated by reference.

In some embodiments, the CXCR4 inhibitor is selected from Ulocuplumab (BMS-936564/MDX1338, Bristol Myers); Motixafortide (BL-8040; BKT-140; BiolineRx); POL6326 (balixafortide, Polyphor); PRX177561 (Proximagen); USL311 (Upsher-Smith Laboratories); Burixafor hydrobromide (TG-0054, TaiGen Biotechnology); LY2510924 (MEDI4736, Eli Lilly); and PF06747143 (Pfizer).

In some embodiments, the CXCR4 inhibitor is selected from mavorixafor; plerixafor (AMD-3100; Sanofi); locuplumab (BMS-936564/MDX1338, Bristol Myers), a fully human anti-CXCR4 antibody, Kashyap el al. (2015) Oncotarget 7:2809-2822; Motixafortide (BL-8040; BKT-140; BiolineRx) Crees et al. (2021) Blood, 138 (Suppl): 475 Abstract 711; POL6326 (balixafortide, Polyphor) Karpova et al. (2017) Journal of Translational Medicine 15:2; PRX177561 (Proximagen) Gravina et al. (2017) Tumor Biology, June 2017:1-17; USL311 (Upsher-Smith Laboratories) NCT02765165; Burixafor hydrobromide (TG-0054, TaiGen Biotechnology) NCT02478125; LY2510924 (MEDI4736, Eli Lilly); NCT02737072, a CXCR4 Cyclic peptide antagonist; PF06747143 (Pfizer), a CXCR4 humanized IgG1 antagonist; BGB—11417; LOXO-338; LP-108; S55746; APG-257; APG-1252 (pelcitoclax); AT-101; TQB3909; obatoclax; GDC-0199; ABT-737; and navitoclax (ABT-263); or a pharmaceutically acceptable salt thereof.

In some embodiments, the CXCR4 inhibitor is mavorixafor, plerixafor, ulocuplumab, motixafortide, POL6326, PRX177561, USL311, burixafor, LY2510924, PF06747143, CX549, BPRCX807, TC14012, USL-311, FC131, CTCE-9908, or GMI 1359.

In some embodiments, the CXCR4 inhibitor is selected from one of the following:

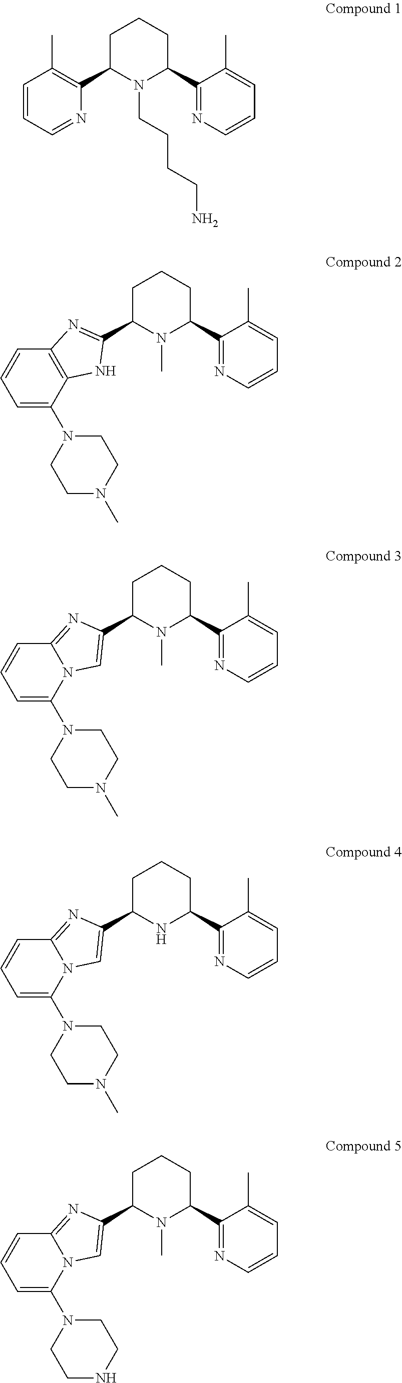 [SMILES: Cc1cccnc1[C@H]1CCC[C@@H](c2ncccc2C)N1CCCCN ; Cc1cccnc1[C@@H]1CCC[C@H](c2nc3cccc(N4CCN(C)CC4)c3[nH]2)N1C ; Cc1cccnc1[C@@H]1CCC[C@H](c2cn3c(N4CCN(C)CC4)cccc3n2)N1C ; Cc1cccnc1[C@@H]1CCC[C@H](c2cn3c(N4CCN(C)CC4)cccc3n2)N1 ; Cc1cccnc1[C@@H]1CCC[C@H](c2cn3c(N4CCNCC4)cccc3n2)N1C]

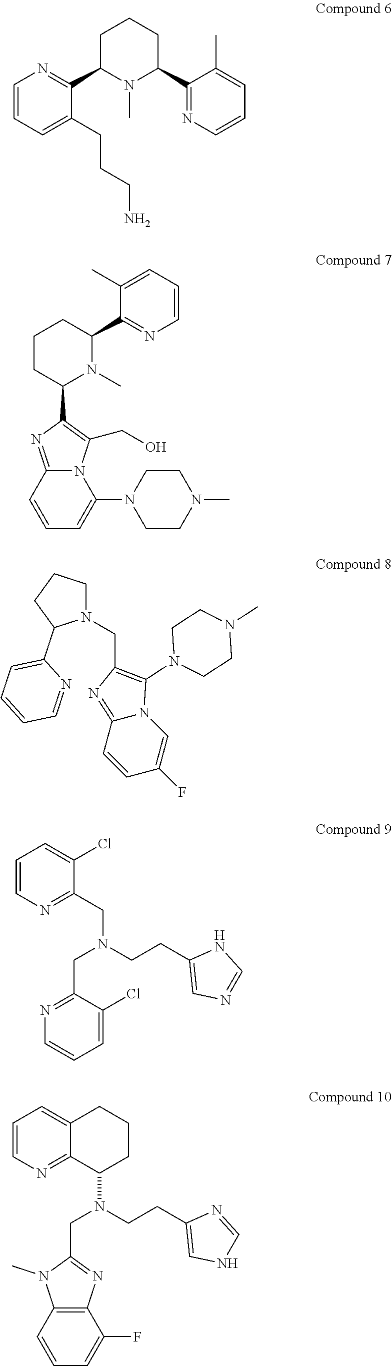

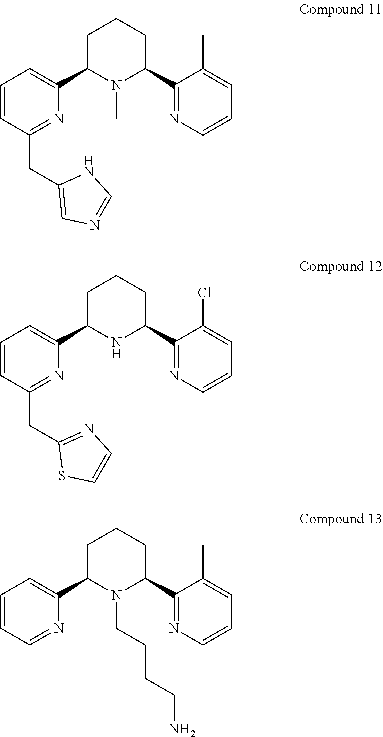 [SMILES: Cc1cccnc1[C@@H]1CCC[C@H](c2cccc(Cc3cnc[nH]3)n2)N1C ; Clc1cccnc1[C@@H]1CCC[C@H](c2cccc(Cc3nccs3)n2)N1 ; Cc1cccnc1[C@@H]1CCC[C@H](c2ccccn2)N1CCCCN]

or a pharmaceutically acceptable salt thereof.

In some embodiments, the CXCR4 inhibitor is mavorixafor or a pharmaceutically acceptable salt thereof

### Mavorixafor as a Single Agent

In one aspect, the present invention provides a method of treating an inflammatory condition that results in increased Interleukin-6 (IL-6) levels in the human body in a patient in need thereof, comprising administering to the patient an effective amount of mavorixafor, or a pharmaceutically acceptable salt thereof.

Mavorixafor (formerly designated X4P-001 and AMD11070) is a CXCR4 antagonist, with molecular formula C₂₁H₂₇N₅; molecular weight 349.48 amu; and appearance as a white to pale yellow solid. Solubility: freely soluble in the pH range 3.0 to 8.0 (>100 mg/mL), sparingly soluble at pH 9.0 (10.7 mg/mL) and slightly soluble at pH 10.0 (2.0 mg/mL). Mavorixafor is only slightly soluble in water. Melting point: 108.9° C.

The chemical structure of mavorixafor is depicted below.

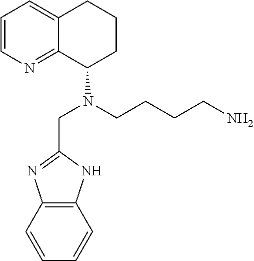 [SMILES: NCCCCN(Cc1nc2ccccc2[nH]1)[C@H]1CCCc2cccnc21]

In certain embodiments, a pharmaceutical composition containing mavorixafor or a pharmaceutically acceptable salt thereof is administered orally in an amount from about 200 mg to about 1200 mg daily. In certain embodiments, the dosage composition may be provided twice a day in divided dosage, approximately 12 hours apart. In other embodiments, the dosage composition may be provided once daily. The terminal half-life of mavorixafor has been generally determined to be between about 12 to about 24 hours, or approximately 14.5 hrs. Dosage for oral administration may be from about 100 mg to about 1200 mg once or twice per day. In certain embodiments, the dosage of mavorixafor or a pharmaceutically acceptable salt thereof useful in the invention is from about 200 mg to about 600 mg daily. In other embodiments, the dosage of mavorixafor or a pharmaceutically acceptable salt thereof useful in the invention may range from about 400 mg to about 800 mg, from about 600 mg to about 1000 mg or from about 800 mg to about 1200 mg daily. In certain embodiments, the invention comprises administration of an amount of mavorixafor or a pharmaceutically acceptable salt thereof of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg.

In some embodiments, a provided method comprises administering to the patient a pharmaceutically acceptable composition comprising mavorixafor or a pharmaceutically acceptable salt thereof wherein the composition is formulated for oral administration. In certain embodiments, the composition is formulated for oral administration in the form of a tablet or a capsule. In some embodiments, the composition comprising mavorixafor or a pharmaceutically acceptable salt thereof is formulated for oral administration in the form of a capsule.

In certain embodiments, a provided method comprises administering to the patient one or more unit doses, such as capsules, comprising 100-1200 mg mavorixafor or a pharmaceutically acceptable salt thereof as an active ingredient; and one or more pharmaceutically acceptable excipients.

A composition according to the present invention comprises a compound for use in the invention or a pharmaceutically acceptable salt or derivative thereof and a pharmaceutically acceptable carrier, adjuvant, or vehicle. The amount of compound in compositions of this invention is an amount effective to measurably IL-6, in a biological sample or in a patient. In certain embodiments, a composition of this invention is formulated for administration to a patient in need of such a composition. In some embodiments, a composition of this invention is formulated for oral administration to a patient.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In certain embodiments, the present invention provides a pharmaceutical composition comprising mavorixafor or a pharmaceutically acceptable salt thereof, one or more diluents, a disintegrant, a lubricant, a flow aid, and a wetting agent. In some embodiments, the present invention provides a composition comprising 10-1200 mg mavorixafor or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In some embodiments, the present invention provides a unit dosage form wherein said unit dosage form comprises a composition comprising 10-200 mg mavorixafor, or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In certain embodiments, the present invention provides a unit dosage form comprising a composition comprising mavorixafor or a pharmaceutically acceptable salt thereof, present in an amount of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg. In some embodiments, a provided composition (or unit dosage form) is administered to the patient once per day, twice per day, three times per day, or four times per day. In some embodiments, a provided composition (or unit dosage form) is administered to the patient once per day or twice per day. In some embodiments, the unit dosage form comprises a capsule containing about 25 mg, about 50 mg, about 75 mg, about 100 mg, about 150 mg, or about 200 mg of mavorixafor or a pharmaceutically acceptable salt thereof.

In some embodiments, the mavorixafor or pharmaceutically acceptable salt thereof is administered in an amount effective to reduce IL-6 overexpression in a patient's cells.

In some embodiments, mavorixafor or a pharmaceutically acceptable salt thereof is administered to the patient in a dose of about 100 mg to about 1000 mg per day.

In some embodiments, mavorixafor or a pharmaceutically acceptable salt thereof is administered to the patient in a dose of about 200 mg to about 600 mg per day.

In some embodiments, mavorixafor or a pharmaceutically acceptable salt thereof is administered to the patient in a dose of about 200 mg, about 400 mg, or about 600 mg per day.

In some embodiments, mavorixafor or a pharmaceutically acceptable salt thereof is administered to the patient in a single daily dose (QD).

In some embodiments, mavorixafor, or a pharmaceutically acceptable salt thereof or pharmaceutical composition thereof, is administered to a patient in a fasted state.

In some embodiments, a CXCR4 inhibitor such as mavorixafor or a pharmaceutically acceptable salt thereof is administered in an amount effective to provide a significant benefit and/or treat patients with inflammatory conditions where IL-6 is overexpressed or hypersecreted, such as Rheumatoid Arthritis (RA), Giant Cell Arteritis (GCA), Polyarticular Juvenile Idiopathic Arthritis (PJIA), Systemic Juvenile Idiopathic Arthritis (SJIA), and Cytokine Release Syndrome (CRS). In some embodiments, mavorixafor or a pharmaceutically acceptable salt thereof is used to treat Rheumatoid Arthritis (RA) in patients with inadequate response to one or more Disease-Modifying Anti-Rheumatic Drugs (DMARDs).

In some embodiments, the inflammatory condition is selected from Rheumatoid Arthritis (RA), Giant Cell Arteritis (GCA), Polyarticular Juvenile Idiopathic Arthritis (PJIA), Systemic Juvenile Idiopathic Arthritis (SJIA), and Cytokine Release Syndrome (CRS).

In some embodiments, the inflammatory condition is Rheumatoid Arthritis (RA) and wherein the patient has had an inadequate response to one or more Disease-Modifying Anti-Rheumatic Drugs (DMARDs). An inadequate response is defined as being not low disease activity at 12 months in more than 3 of 4 composite measures, as described by Aramaki et. al (Mod Rheumatol. 2020 January; 30(1):50-57. (pubmed.ncbi.nlm.nih.gov/30482075/); hereby incorporated by reference.

A CXCR4 inhibitor such as mavorixafor can also be used to treat Systemic Sclerosis-Associated Interstitial Lung Disease (SSc-ILD); septic shock; chronic ABMR [antibody-mediated rejection]; Adult Onset Still Disease (AOSD); Neuromyelitis optica spectrum disorder (NMOSD); and Multicentric Castleman's disease (MCD) (in patients who are human immunodeficiency virus (HIV) negative and human herpesvirus-8 (HHV-8) negative).

Multicentric Castleman's disease (MCD) (in patients who are human immunodeficiency virus (HIV) negative and human herpesvirus-8 (HHV-8) negative).

In some embodiments, the inflammatory condition is selected from Systemic Sclerosis-Associated Interstitial Lung Disease (SSc-ILD); septic shock; chronic ABMR [antibody-mediated rejection]; Adult Onset Still Disease (AOSD); Neuromyelitis optica spectrum disorder (NMOSD); and Multicentric Castleman's disease (MCD). In some embodiments, the inflammatory condition is Multicentric Castleman's disease (MCD) and the patient is human immunodeficiency virus (HIV) negative and human herpesvirus-8 (HHV-8) negative.

In some embodiments, mavorixafor or a pharmaceutically acceptable salt thereof or pharmaceutical composition thereof is used instead of an approved anti IL-6 therapy such as anti-IL-6 receptor monoclonal antibodies (mAbs) (e.g., sarilumab, tocilizumab) or an anti-IL-6 ligand antibody (i.e., siltuximab). In some embodiments, mavorixafor or a pharmaceutically acceptable salt thereof or pharmaceutical composition thereof is used instead of anti-IL-6 receptor mAbs currently in clinical trials, including olokizumab, elsilimomab, clazakizumab, sirukumab, levilimab, and CPSI-2364. In some embodiments, mavorixafor or a pharmaceutically acceptable salt thereof or pharmaceutical composition thereof is used instead of an anti-IL-6 receptor mAbs currently in preclinical trials, such as include ARGX-109, FE301, and FM101.

In some embodiments, mavorixafor or a pharmaceutically acceptable salt thereof or pharmaceutical composition thereof is used instead of Tocilizumab, Sarilumab, Satralizumab, Vobarilizumab, Siltuximab, olokizumab, sirukumab, elsilimomab, clazakizumab, levilimab, CPSI-2364, ARGX-109, FE301, or FM101 for the suppression of IL-6.

In some embodiments, mavorixafor or a pharmaceutically acceptable salt thereof or pharmaceutical composition thereof is used as a single therapeutic agent to treat Rheumatoid Arthritis (RA), Giant Cell Arteritis (GCA), Polyarticular Juvenile Idiopathic Arthritis (PJIA), Systemic Juvenile Idiopathic Arthritis (SJIA), and Cytokine Release Syndrome (CRS), Systemic Sclerosis-Associated Interstitial Lung Disease (SSc-ILD); septic shock; chronic ABMR [antibody-mediated rejection]; Adult Onset Still Disease (AOSD); Neuromyelitis optica spectrum disorder (NMOSD); and Multicentric Castleman's disease (MCD) (in patients who are human immunodeficiency virus (HIV) negative and human herpesvirus-8 (HHV-8) negative).

### Mavorixafor in Combination Therapy

In one aspect, the present invention provides a method of treating an inflammatory condition that results in increased Interleukin-6 (IL-6) levels in the human body in a patient in need thereof, comprising administering to the patient an effective amount of mavorixafor, or a pharmaceutically acceptable salt thereof, in combination with an effective amount of IL-6 inhibitor.

In some embodiments, the IL-6R inhibitor is an IL-6 receptor antibody or an IL-6 ligand antibody.

In some embodiments, mavorixafor or a pharmaceutically acceptable salt thereof or pharmaceutical composition thereof is used in combination with an approved anti IL-6 therapy such as anti-IL-6 receptor monoclonal antibodies (mAbs) such as sarilumab, tocilizumab, and satralizumab, or an anti-IL-6 ligand antibody such as siltuximab. In some embodiments, mavorixafor or a pharmaceutically acceptable salt thereof or pharmaceutical composition thereof is used in combination with anti-IL-6 receptor mAbs currently in clinical trials, including vobarilizumab, olokizumab, elsilimomab, clazakizumab, sirukumab, levilimab, Ziltivekimab, Avidia, and CPSI-2364. In some embodiments, mavorixafor or a pharmaceutically acceptable salt thereof or pharmaceutical composition thereof is used in combination with anti-IL-6 receptor mAbs currently in preclinical trials, including ARGX-109, FE301, and FM101.

In some embodiments, mavorixafor or a pharmaceutically acceptable salt thereof or pharmaceutical composition thereof is used in combination with a second therapeutic agent selected from olokizumab, elsilimomab, clazakizumab, sirukumab, levilimab, CPSI-2364, ARGX-109, FE301, and FM101.

In some embodiments, mavorixafor or a pharmaceutically acceptable salt thereof or pharmaceutical composition thereof is used in combination with tocizumab (ACTEMRA®).

With the reduction of available CXCR4 for binding by CXCL12 it is now theorized that, because CXCR4 overexpression is mechanistically-related to IL-6 hypersecretion via the CXCR4/CXCL12/IL-6 axis, CXCR4 inhibitors, such as mavorixafor may be effective for treatment and prevention of conditions characterized by overexpression of IL-6, including rheumatoid arthritis and related disorders, and cytokine release syndrome (CRS) which may be induced by treatment with chimeric antigen receptors in recombinant T cell therapies such as tisagenlecleucel (KYMRIAH®, Novartis) or axicabtagene ciloleucel (YESCARTA®, Kite/Gilead).

In one aspect, the present invention provides a method of treating an inflammatory condition in a patient in need thereof, comprising administering to the patient an effective amount of mavorixafor, or a pharmaceutically acceptable salt thereof, in combination with an effective amount of an IL-6 inhibitor.

In some embodiments, the IL-6 inhibitor is selected from Tocilizumab (ACTEMRA® Chugai Seiyaku Kabushiki Kaisha Corp./Roche, Genentech); Sarilumab (Kevzara®, Sanofi/Regeneron); and Satralizumab (Enspryng® Chugai and Roche).

In some embodiments, the IL-6 inhibitor is selected from Vobarilizumab (ALX-0061 Ablynx); Siltuximab (SYLVANT®, EUSA Pharma); Olokizumab (CDP6038, UCB Pharma); Sirukumab (CNTO-136; Janssen/JNJ); and Clazakizumab (formerly ALD518 and BMS-945429).

In some embodiments, the IL-6 inhibitor is Tocilizumab.

In some embodiments, the IL-6 inhibitor is Sarilumab.

In some embodiments, the IL-6 inhibitor is Satralizumab.

In some embodiments, the inflammatory condition is selected from rheumatoid arthritis, Giant cell arteritis, Polyarticular Juvenile Idiopathic Arthritis, Systemic Juvenile Idiopathic Arthritis, and Cytokine Release Syndrome.

Additional agents that may be co-administered with mavorixafor are described in WO 2018/237158, the entire contents of which are hereby incorporated by reference.

The IL-6 inhibitor may be simultaneously or sequentially administered with the mavorixafor or a pharmaceutically acceptable salt thereof. In some embodiments, the mavorixafor and the IL-6 inhibitor are co-administered, for example, as part of the same pharmaceutical composition. In some embodiments, the mavorixafor and the IL-6 inhibitor are co-administered as part of separate pharmaceutical compositions.

In some embodiments, an IL-6 inhibitor is co-administered with mavorixafor or a pharmaceutically acceptable salt thereof. In some embodiments, the IL-6 inhibitor is Tocilizumab, Sarilumab, or Satralizumab; or a pharmaceutically acceptable salt thereof.

Tocilizumab (ACTEMRA® Chugai Seiyaku Kabushiki Kaisha Corp./Roche, Genentech) is an interleukin-6 (IL-6) receptor antagonist indicated for treatment of: (1) Adult patients with moderately to severely active rheumatoid arthritis (RA) who have had an inadequate response to one or more Disease-Modifying Anti-Rheumatic Drugs (DMARDs); (2) Adult patients with giant cell arteritis (GCA); (3) Patients 2 years of age and older with active poly-articular juvenile idiopathic arthritis (PJIA); (4) Patients 2 years of age and older with active systemic juvenile idiopathic arthritis (SJIA); and (5) Adults and pediatric patients 2 years of age and older with chimeric antigen receptor (CAR) T cell-induced severe or life-threatening cytokine release syndrome (CRS).

It is recommended that tocilizumab not be initiated in patients with an absolute neutrophil count (ANC) below 2000 per mm³, platelet count below 100,000 per mm³, or who have ALT or AST above 1.5 times the upper limit of normal (ULN). Patients with severe or life-threatening CRS frequently have cytopenias or elevated ALT or AST due to the lymphodepleting chemotherapy or the CRS. The decision to administer tocilizumab should take into account the potential benefit of treating the CRS versus the risks of short-term treatment with tocilizumab.

In the United States, Tocilizumab may be administered for the treatment of Rheumatoid Arthritis (RA) in adult patients with moderately to severely active rheumatoid arthritis who have had an inadequate response to one or more Disease-Modifying Anti-Rheumatic Drugs (DMARDs). For RA, tocilizumab may be administered as monotherapy or concomitantly with methotrexate or other non-biologic DMARDs. For RA, the recommended dosage regimen, given as a 60-minute single intravenous drip infusion, is 4 mg per kg every 4 weeks followed by an increase to 8 mg per kg every 4 weeks based on clinical response. Reduction of dose from 8 mg per kg to 4 mg per kg is recommended for management of certain dose-related laboratory changes including elevated liver enzymes, neutropenia, and thrombocytopenia. Doses exceeding 800 mg per infusion are not recommended in RA patients. The recommended dosage for subcutaneous injection is 162 mg administered subcutaneously every other week, followed by an increase to every week based on clinical response for patients less than 100 kg body weight. For patients of 100 kg or more body weight, the recommended dosage for subcutaneous injection is 162 mg administered subcutaneously every week.

In the United States, Tocilizumab may be administered for the treatment of Giant Cell Arteritis (GCA) in adult patients with giant cell arteritis. The recommended dose for adult patients with GCA is 162 mg given once every week as a subcutaneous injection in combination with a tapering course of glucocorticoids. Based on clinical considerations, a dose of 162 mg given once every other week as a subcutaneous injection in combination with a tapering course of glucocorticoids may be prescribed.

In the United States, Tocilizumab may be administered for the treatment of Polyarticular Juvenile Idiopathic Arthritis (PJIA) in patients 2 years of age and older with active poly-articular juvenile idiopathic arthritis. For patients with PJIA, tocilizumab may be used alone or in combination with methotrexate. The recommended dosage for PJIA patients given once every 4 weeks as a 60-minute single intravenous drip infusion is 10 mg/kg for patients of less than 30 kg body weight and 8 mg/kg for patients at or above 30 kg body weight.

In the United States, Tocilizumab may be administered for the treatment of Systemic Juvenile Idiopathic Arthritis (SJIA) in patients 2 years of age and older with active systemic juvenile idiopathic arthritis. For patients with SJIA, tocilizumab may be used alone or in combination with methotrexate.

The recommended dosage for SJIA patients given once every 2 weeks as a 60-minute single intravenous drip infusion is 12 mg/kg for patients of less than 30 kg body weight and 8 mg/kg for patients at or above 30 kg body weight.

In the United States, Tocilizumab may be administered for the treatment of Cytokine Release Syndrome (CRS) in adults and pediatric patients 2 years of age and older with chimeric antigen receptor (CAR) T cell-induced severe or life-threatening cytokine release syndrome. For treatment of CRS, intravenous infusion of tocilizumab is to be administered as a 60-minute intravenous infusion at a recommended dosage of 12 mg/kg for patients of less than 30 kg body weight and 8 mg/kg for patients at or above 30 kg body weight. If no clinical improvement in the signs and symptoms of CRS occurs after the first dose, up to 3 additional doses of tocilizumab may be administered. The interval between consecutive doses should be at least 8 hours. Doses exceeding 800 mg per infusion are not recommended in CRS patients.

Tocilizumab is provided for intravenous infusion by injection: 80 mg/4 mL, 200 mg/10 mL, 400 mg/20 mL as a clear, colorless to pale yellow solution in 20 mg/mL single-dose vials for further dilution prior to intravenous infusion. For subcutaneous injection tocilizumab is given at a dosage of 162 mg/0.9 mL clear, colorless to slightly yellowish solution in a single-dose prefilled syringe.

In the United States, Sarilumab (Kevzara®, Sanofi/Regeneron) is a recombinant humanized anti-IL-6 receptor mAb that is indicated for treatment of adult patients with moderately to severely active rheumatoid arthritis who have had an inadequate response or intolerance to one or more disease-modifying antirheumatic drugs (DMARDs). It acts to inhibit binding between IL-6 and IL-6R. Sarilumab may be used as monotherapy or in combination with methotrexate (MTX) or other conventional DMARDs. The recommended dosage of sarilumab is 200 mg once every two weeks, administered as a subcutaneous injection, with dosage modification to manage neutropenia, thrombocytopenia, and/or elevated liver transaminases. Sarilumab initiation is not recommended in patients with ANC less than 2000/mm3, platelets less than 150,000/mm3 or liver transaminases above 1.5 times ULN. Sarilumab is provided as a subcutaneous formulation for injection of 150 mg/1.14 mL or 200 mg/1.14 mL solution in a single-dose pre-filled syringe or pen. Sarilumab is not approved for the treatment of cytokine release syndrome.

In the United States, Satralizumab (Enspryng® Chugai and Roche) is a humanized anti-interleukin-6 (IL-6) receptor monoclonal recycling antibody indicated for the treatment of neuromyelitis optica spectrum disorder (NMOSD) in adult patients who are anti-aquaporin-4 (AQP4) antibody positive. Hepatitis B virus, tuberculosis, and liver transaminase screening is required before the first dose. Prior to every use, determine if there is an active infection. The recommended loading dosage of satralizumab for the first three administrations is 120 mg by subcutaneous injection at Weeks 0, 2, and 4, followed by a maintenance dosage of 120 mg every 4 weeks. Satralizumab is provided as a subcutaneous formulation for injection of 120 mg/mL in a single-dose prefilled syringe. It is recommended to delay satralizumab administration in patients with an active infection until the infection is resolved. Vaccination with live or live-attenuated vaccines is not recommended during treatment. Patients should be monitored for elevated levels of liver enzymes ALT and AST during treatment; interruption of satralizumab may be required. Patients should also be monitored for decreased neutrophil counts during treatment.

In certain embodiments, mavorixafor or a pharmaceutically acceptable salt thereof, or another CXCR4 antagonist, is administered in combination with an additional therapeutic agent. In some embodiments, mavorixafor or a pharmaceutically acceptable salt thereof, or another CXCR4 antagonist, is administered in combination with one additional therapeutic agent. In some embodiments, mavorixafor or a pharmaceutically acceptable salt thereof, or another CXCR4 antagonist, is administered in combination with two additional therapeutic agents. In some embodiments, mavorixafor or a pharmaceutically acceptable salt thereof, or another CXCR4 antagonist, is administered in combination with three or more additional therapeutic agents.

In some embodiments, the dose of the IL-6 inhibitor required for effective treatment is decreased by at least 20% relative to the effective dose of the IL-6 inhibitor as a monotherapy.

In some embodiments, the dose of the IL-6 inhibitor required for effective treatment is decreased by at least 25% relative to the effective dose of the IL-6 inhibitor, or a pharmaceutically acceptable salt thereof, as a monotherapy.

In some embodiments, the dose of the IL-6 inhibitor required for effective treatment is decreased by at least 30% relative to the effective dose of the IL-6 inhibitor, or a pharmaceutically acceptable salt thereof, as a monotherapy.

In some embodiments, the dose of the IL-6 inhibitor required for effective treatment is decreased by at least 35% relative to the effective dose of the IL-6 inhibitor, or a pharmaceutically acceptable salt thereof, as a monotherapy.

In some embodiments, the dose of the IL-6 inhibitor required for effective treatment is decreased by at least 40% relative to the effective dose of the IL-6 inhibitor, or a pharmaceutically acceptable salt thereof, as a monotherapy.

In some embodiments, the dose of the IL-6 inhibitor required for effective treatment is decreased by at least 50% relative to the effective dose of the IL-6 inhibitor, or a pharmaceutically acceptable salt thereof, as a monotherapy.

In some embodiments, the dose of the IL-6 inhibitor required for effective treatment is decreased by at least 60% relative to the effective dose of the IL-6 inhibitor, or a pharmaceutically acceptable salt thereof, as a monotherapy.

In some embodiments, the dose of the IL-6 inhibitor required for effective treatment is decreased by at least 70% relative to the effective dose of the IL-6 inhibitor, or a pharmaceutically acceptable salt thereof, as a monotherapy.

In some embodiments, the dose of the IL-6 inhibitor required for effective treatment is decreased by at least 80% relative to the effective dose of the IL-6 inhibitor, or a pharmaceutically acceptable salt thereof, as a monotherapy.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions. Thus the kit of the invention includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

The contents of each document cited in the specification are herein incorporated by reference in their entireties.

### EXEMPLIFICATION

### Example 1: Mavorixafor Reduces IL-6 in WM Cells

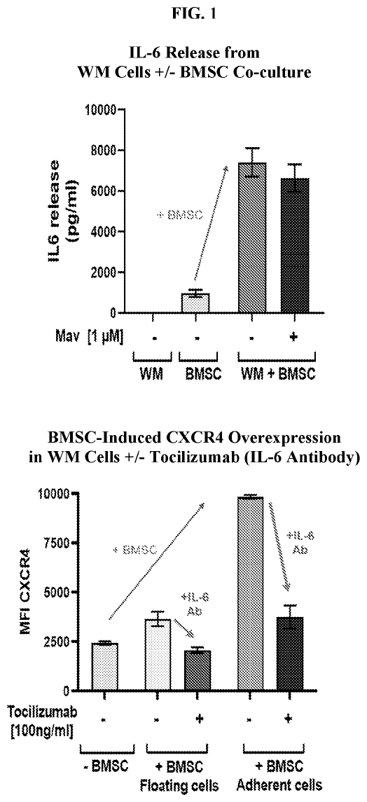

As shown in FIG. 1, a co-culture of bone marrow stroma cells (BMSC) with Waldenström's macroglobulinemia (WM) cells induces IL-6 hypersecretion in BMSC and CXCR4 overexpression in WM Cells.

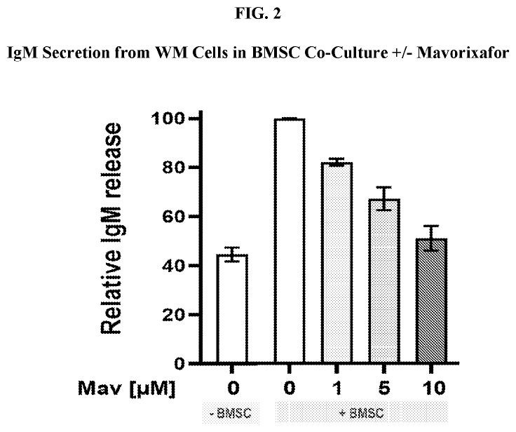

As shown in FIG. 2, Mavorixafor blocks bone marrow stroma-induced IgM hypersecretion in WM Cells.

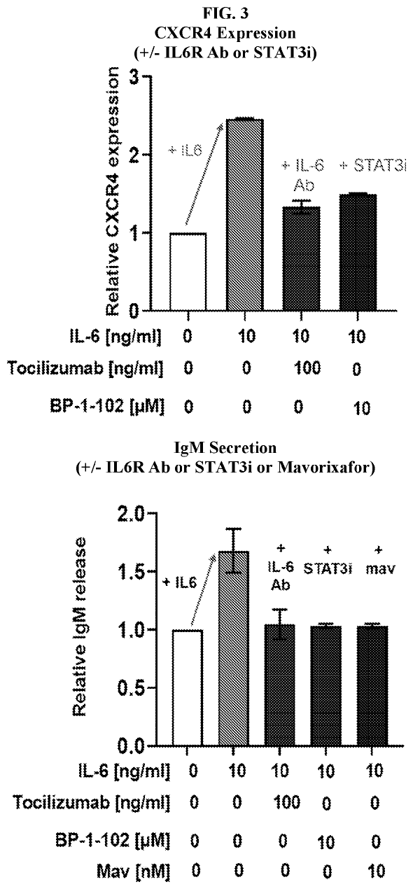

As shown in FIG. 3, even in the absence of BMSC, soluble IL-6 upregulates CXCR4 Expression and IgM Secretion in WM Cells; this upregulation is prevented by blockade of IL-6/IL6R/STAT3 axis.

### REFERENCES

1. Buske C, Leblond V, Dimopoulos M, et al. Waldenstrom's macroglobulinaemia: ESMO Clinical Practice Guidelines for diagnosis, treatment and follow-up. Ann Oncol. 2013; 24 (6): vi155-9.
2. Cao Y, Hunter Z, Liu X, et al. The WHIM-like CXCR4(S338X) somatic mutation activates AKT and ERK, and promotes resistance to ibrutinib and other agents used in the treatment of Waldenstrom's Macroglobulinemia. Leukemia. 2014; 29(1): 169-76.
3. Cao Y, Hunter Z, Liu X, et al. CXCR4 WHIM-like frameshift and nonsense mutations promote ibrutinib resistance but do not supplant MYD88(L265P)-directed survival signaling in Waldenstrom macroglobulinaemia cells. Br J Haematol. 2015; 168(5): 701-7.
4. Castillo J, Olszewski A, Kanan S, et al. Overall survival and competing risks of death in patients with Waldenstrom macroglobulinaemia: an analysis of the Surveillance, Epidemiology and End Results database. Br J Haematol. 2015; 169(1): 81-9.
5. Chatterjee S, Azad B, Nimmagadda S. The intricate role of CXCR4 in cancer. Adv Cancer Res. 2014; 124: 31-82.
6. Christiansen C F, Johansen M B, Langeberg W J, et al. Incidence of acute kidney injury in cancer patients: a Danish population-based cohort study. Eur J Intern Med. 2011; 4: 399-406.
7. Dale D, Bolyard A, Kelley M, et al. The CXCR4 antagonist plerixafor is a potential therapy for myelokathexis, WHIM syndrome. Blood. 2011; 118(18): 4963-66. doi: 10.11.1182/blood-2011-06-360586
8. de Duve C, de Barsy T, Poole B, et al. Lysosomotropic agents. Biochem Pharmacol. 1974; 23: 2495-2534.
9. De Falco V, Guarino V, Avilla E, et al. Biological role and potential therapeutic targeting of the chemokine receptor CXCR4 in undifferentiated thyroid cancer. Cancer Res 2007; 67: 11821-9.
10. Dimopoulos M, Panayiotidis P, Moulopoulos L, et al. Waldenstrom's macroglobulinemia: clinical features, complications, and management. J Clin Oncol. 2000; 18(1): 214-26.
11. Dimopoulos M, Anagnostopoulos A, Kyrtsonis M, et al. Primary treatment of Waldenstrom macroglobulinemia with dexamethasone, rituximab, and cyclophosphamide. J Clin Oncol. 2007; 25(22): 3344-9.
12. Dimopoulos M, Kastritis E, Owen R, et al. Treatment recommendations for patients with Waldenstrom macroglobulinemia (WM) and related disorders: IWWM-7 consensus. Blood. 2014; 124(9): 1404-11.
13. Dimopoulos M, Trotman J, Tedeschi A, et al. Ibrutinib for patients with rituximab-refractory Waldenstrom's macroglobulinaemia (INNOVATE): an open-label substudy of an international, multicentre, Phase 3 trial. Lancet Oncol. 2017; 18(2): 241-250.
14. Dimopoulos M, Tedeschi A, Trotman J, et al. Phase 3 Trial of Ibrutinib plus Rituximab in Waldenstrom's Macroglobulinemia. N Engl J Med. 2018; 378(25): 2399-2410.
15. FDA Drug development and drug interactions: table of substrates, inhibitors and inducers. Updated Nov. 14, 2017. Available at fda.gov/drugs/drug-interactions-labeling/drug-development-and-drug-interactions-table-substrates-inhibitors-and-inducers. Accessed Jun. 22, 2019.
16. Fonseca R, Hayman S. Waldenstrom macroglobulinaemia. Br J Haematol. 2007; 138(6): 700-20.
17. Goldman J, Mendenhall M, Rettinger S. Hyperglycemia associated with targeted oncologic treatment: mechanisms and management. The Oncologist. 2016; 21: 1326-36.
18. Groves F, Travis L, Devesa S, et al. Waldenstrom's macroglobulinemia: incidence patterns in the United States, 1988-1994. Cancer. 1998; 82(6): 1078-81.
19. Hassan S, Buchanan M, Jahan K, et al. CXCR4 peptide antagonist inhibits primary breast tumor growth, metastasis and enhances the efficacy of anti-VEGF treatment or docetaxel in a transgenic mouse model. Int J Cancer 2010; 129: 225-32.
20. Hendrix C, Flexner C, MacFarland R, et al. Pharmacokinetics and safety of AMD-3100, a novel antagonist of the CXCR-4 cytokine receptor, in human volunteers. Antimicrob Agent and Chemother. 2000; 44(6): 1167-73.
21. Hruban Z. Pulmonary changes induced by amphophilic drugs. Environ Health Perspect. 1976; 16: 111-18
22. Huang E H, Singh B, Cristofanilli M, et al. A CXCR4 antagonist CTCE-9908 inhibits primary tumor growth and metastasis of breast cancer. J Surg Res 2009; 155: 231-6.
23. Hunter Z, Xu L, Yang G, et al. The genomic landscape of Waldenstrom macroglobulinemia is characterized by highly recurring MYD88 and WHIM-like CXCR4 mutations, and small somatic deletions associated with B-cell lymphomagenesis. Blood. 2014; 123(11): 1637-46.
24. IMBRUVICA® [Highlights of Prescribing Information]. Horsham, P A: Janssen Biotech, Inc; 2019. imbruvica.com/docs/librariesprovider7/default-document-library/prescribing-information.pdf
25. Ji Y, Jin J, Hyman D, et al. Challenges and opportunities in dose finding in oncology and immuno-oncology. Clin Transl Sci. 2018; 11: 345-51.
26. Jiang H, Passarelli P, Munro G, et al. High-resolution sub-cellular imaging by correlative NanoSIMS and electron microscopy of amiodorone internalisation by lung macrophages as evidence for drug-induced phospholipidosis. Chem Commun. 2017; 53: 1506-1509.
27. Kim S Y, Lee C H, Midura B V, et al. Inhibition of the CXCR4/CXCL12 chemokine pathway reduces the development of murine pulmonary metastases. Clin Exp Metastasis 2008; 25: 201-11.
28. Kodovanti U, Mehendale H. Cationic amphiphilic drugs and phospholipid storage disorder. Pharmcol Review. 1990; 42(4): 327-54.
29. Kyle R A, Therneau T M, Rajkumar S V, et at. Long-term follow-up of 241 patients with monoclonal gammopathy of undetermined significance: the original Mayo Clinic series 25 years later. Mayo Clin Proc. 2004; 79: 859-65.
30. Kyle R A, Rajkumar S V. Monoclonal gammopathy of undetermined significance. Clin Lymphoma Myeloma. 2005; 6: 102-14.
31. Lahoti A, Humphreys B. Chapter 3 AKI associated with malignancies. Amer Soc Nephrol. 2016; 3: 1-8.
32. Lei N, Rubin E, Mehrotra N, et al. Rendering the 3+3 design to rest: more efficient approaches to oncology dose-finding trials in the era of targeted therapy. Clin Cancer Resch. 2016; 22(11): 2623-9.
33. Liu Q, Li Z, Gao J L, et al. CXCR4 antagonist AMD3100 redistributes leukocytes from primary immune organs to secondary immune organs, lung, and blood in mice. Eur J Immunol. 2015: 45(6): 1855-67.
34. Liu Q, Pan C, Lopez L, et al. WHIM syndrome caused by Waldenstrom's macroglobulinemia-associated mutation CXCR4 (L329fs). J Clin Immunol. 2016; 36(4): 397-405.
35. Majumdar S, Murphy P M. Adaptive immunodeficiency in WHIM syndrome. Int J Mol Sci. 2018; 20(1): 3. Published 2018. doi:103390/ijms20010003
36. Maker A V, Yang, J C, Sherry R M, et al. Intrapatient dose escalation of anti-CTLA-4 antibody in patients with metastatic melanoma. J Immunother. 2006; 29(4): 455-463.
37. Mazzucchelli M, Frustaci A, Deodato M, et al. Waldenstrom's macroglobulinemia: an update. Mediterr J Hematol Infect Dis. 2018; 10 (1) e2018004. doi:10.4084/MJHID.2018.004
38. Moyle G, DeJesus E, Boffito M, et al. Proof of activity with AMD11070, an orally bioavailable inhibitor of CXCR4 tropic HIV type 1. Clin Infect Dis. 2009; 48(6): 798-805.
39. Muller A, Homey B, Soto H, et al. Involvement of chemokine receptors in breast cancer metastasis. Nature 2001; 410: 50-6.
40. Murugan R, Karajala-Subramanyam V, Lee M, et al. Acute Kidney injury in non-severe pneumonia is associated with an increased immune response and lower survival. Kidney Int. 2010; 77(6):527-35. doi:10.1038/ki.2009.502
41. Nagase H, Miyamasu M, Yamaguchi M, et al. Expression of CXCR4 in eosinophils: functional analyses and cytokine-mediated regulation. J Immunol. 2000; 164(11):5935-43.
42. Nowakowski G S, Witzig T E, Dinghi D, et al. Circulating plasma cells detected by flow cytometry as a predictor of survival in 302 patients with newly diagnosed multiple myeloma. Blood. 2005; 106: 2276-79.
43. Nyunt M, Becker S, MacFarland R, et al. Pharmacokinetic Effect of AMD070, an Oral CXCR4 Antagonist, on CYP3A4 and CYP2D6 Substrates Midazolam and Dextromethorphan in Healthy Volunteers. J Acquir Immune Defic Syndr. 2008; 47(5):559-565.
44. Olszewski A, Treon S, Castillo J. Evolution of Management and Outcomes in Waldenstrom Macroglobulinemia: A Population-Based Analysis. Oncologist. 2016; 21(11): 1377-1386.
45. Owen R, Treon S, Al-Katib A, et al. Clinicopathological definition of Waldenstrom's macroglobulinemia: consensus panel recommendations from the Second International Workshop on Waldenstrom's Macroglobulinemia. Semin Oncol. 2003; 30(2): 110-5.
46. Owen R, Pratt G, Auer R, et al. Guidelines on the diagnosis and management of Waldenstrom macroglobulinaemia. Br J Haematol. 2014; 165(3): 316-33.
47. Porvasnik S, Sakamoto N, Kusmartsev S, et al. Effects of CXCR4 antagonist CTCE-9908 on prostate tumor growth. Prostate 2009; 69: 1460-9.
48. Pophali P, Bartley A, Kapoor P, et al. Prevalence and survival of smoldering Waldenstrom macroglobulinaemia in the United States. Br J Haematol. 2018. 1011-70.
49. Poulain S, Roumier C, Venet-Caillault A, et al. Genomic Landscape of CXCR4 Mutations in Waldenstrom Macroglobulinemia. Clin Cancer Res. 2016; 22(6): 1480-8.
50. Roccaro A, Sacco A, Himenez C, et al. C1013G/CXCR4 acts as a driver mutation of tumor progression and modulator of drug resistance in lymphoplasmacytic lymphoma. Blood. 2014; 123(26):4120-31.
51. Rubin J B, Kung A L, Klein R S, et al. A small-molecule antagonist of CXCR4 inhibits intracranial growth of primary brain tumors. Proc Nat Acad Sci USA 2003; 100: 13513-8.
52. Sacco A, Fenotti A, Affo, L, et al. The importance of the genomic landscape in Waldenstrom's macroglobulinemia of targeted therapeutical intervention. Oncotarget. 2017; 8: 35435-44.
53. Sarma J S M, Pei H, Venkataraman K. Role of oxidative stress in amiodorone-induced toxicity. J Cardiovasc Pharmacol Therapeut. 1997; 2(1): 53-60.
54. Schimanski C C, Bahre R, Gockel I, et al. Dissemination of hepatocellular carcinoma is mediated via chemokine receptor CXCR4. Br J Cancer 2006; 95: 210-7.
55. Smith M C, Luker K E, Garbow J R, et al. CXCR4 regulates growth of both primary and metastatic breast cancer. Cancer Res 2004; 64: 8604-12.
56. Stone N, Dunaway S, Flexner C, et al. Multiple-dose escalation study of the safety, pharmacokinetics and biologic activity of oral AMD070, a selective CXCR4 receptor inhibitor, in human subjects. Antimicrob Agent and Chemother. 2007; 51(7): 2351-58/-73.
57. Treon S, XIII. Waldenstrom's macroglobulinaemia: an indolent B-cell lymphoma with distinct molecular and clinical features. Hematol Oncol. 2013; 31 (1): 76-80.
58. Treon S, Cao Y, Xu L, et al. Somatic mutations in MYD88 and CXCR4 are determinants of clinical presentation and overall survival in Waldenstrom macroglobulinemia. Blood. 2014; 123(18): 2791-6.
59. Treon S, Tripsas C, Meid K, et al. Ibrutinib in previously treated Waldenstrom's macroglobulinemia. N Engl J Med. 2015; 372(15): 1430-40. [1]
60. Treon S, How I treat Waldenstrom macroglobulinemia. Blood. 2015; 126(6): 721-32. [2]
61. Treon S, Gustine J, Meid K, et al. Ibrutinib monotherapy in symptomatic, treatment-naive patients with Waldenstrom macroglobulinemia. J Clin Oncol. 2018; 36(27): 2755-2761. [1]
62. Treon S, Meid K, Gustine J, et al. Ibrutinib shows prolonged progression-free survival in symptomatic, previously treated patients with MYD88 mutated Waldenstrom's macroglobulinemia: long-term follow-up of pivotal trial (NCT01614821). Abstr. PS1185. 23rd Conf. Euro Hem Assoc. Jun. 16, 2018. Stockholm, Sweden. [2]
63. Varettoni M, Zibellini S, Defrancesco I, et al. Pattern of somatic mutations in patients with Waldenstrom macroglobulinemia or IgM monoclonal gammopathy of undetermined significance. Haematologica. 2017; 102(12): 2077-2085.
64. Xu L, Hunter Z, Tsakmaklis N, et al. Clonal architecture of CXCR4 WHIM-like mutations in Waldenstrom Macroglobulinaemia. Br J Haematol. 2016; 172(5): 735-44.
65. Zeelenberg I S, Ruuls-Van Stalle L E, Roos E. The chemokine receptor CXCR4 is required for outgrowth of colon carcinoma micrometastases. Cancer Res 2003; 63: 3833-9.

We claim:

1. A method of treating an inflammatory condition that results in increased Interleukin-6 (IL-6) levels in the human body in a patient in need thereof, comprising administering to the patient an effective amount of mavorixafor, or a pharmaceutically acceptable salt thereof.

2. The method of claim 1, wherein the inflammatory condition is selected from Rheumatoid Arthritis (RA), Giant Cell Arteritis (GCA), Polyarticular Juvenile Idiopathic Arthritis (PJIA), Systemic Juvenile Idiopathic Arthritis (SJIA), and Cytokine Release Syndrome (CRS).

3. The method of claim 2, wherein the inflammatory condition is Rheumatoid Arthritis (RA) and wherein the patient has had an inadequate response to one or more Disease-Modifying Anti-Rheumatic Drugs (DMARDs).

4. The method of claim 1, wherein the inflammatory condition is selected from Systemic Sclerosis-Associated Interstitial Lung Disease (SSc-ILD); septic shock; chronic ABMR [antibody-mediated rejection]; Adult Onset Still Disease (AOSD); Neuromyelitis optica spectrum disorder (NMOSD); and Multicentric Castleman's disease (MCD).

5. The method of claim 4, wherein the inflammatory condition is Multicentric Castleman's disease (MCD) and the patient is human immunodeficiency virus (HIV) negative and human herpesvirus-8 (HHV-8) negative.

6. The method of any one of claims 1-5, wherein the mavorixafor or a pharmaceutically acceptable salt thereof is administered orally in an amount from about 200 mg to about 1200 mg daily.

7. The method of claim 6, wherein the amount of mavorixafor or a pharmaceutically acceptable salt thereof is about 200 mg to about 600 mg daily.

8. The method of claim 6 or claim 7, wherein the mavorixafor or a pharmaceutically acceptable salt thereof is administered twice a day in divided dosage, approximately 12 hours apart.

9. The method of claim 6 or claim 7, wherein the mavorixafor or a pharmaceutically acceptable salt thereof is administered once daily.

10. A method of treating an inflammatory condition that results in increased Interleukin-6 (IL-6) levels in the human body in a patient in need thereof, comprising administering to the patient an effective amount of mavorixafor, or a pharmaceutically acceptable salt thereof, in combination with an IL-6R inhibitor.

11. The method of claim 10, wherein the IL-6R inhibitor is an IL-6 receptor antibody or an IL-6 ligand antibody.

12. The method of claim 10 or claim 11, wherein the IL-6R inhibitor is selected from sarilumab, tocilizumab, satralizumab, and siltuximab.

13. The method of claim 12, wherein the IL-6R inhibitor is tocilizumab.

14. The method of claim 10, wherein the TL-6R inhibitor is selected from vobarilizumab, olokizumab, elsilimomab, clazakizumab, sirukumab, levilimab, ziltivekimab, avidia, and CPSI-2364.

15. The method of claim 10, wherein the inflammatory condition is selected from Rheumatoid Arthritis (RA), Giant Cell Arteritis (GCA), Polyarticular Juvenile Idiopathic Arthritis (PJIA), Systemic Juvenile Idiopathic Arthritis (SJIA), and Cytokine Release Syndrome (CRS).

16. The method of claim 15, wherein the inflammatory condition is Rheumatoid Arthritis (RA) and wherein the patient has had an inadequate response to one or more Disease-Modifying Anti-Rheumatic Drugs (DMARDs).

17. The method of claim 10, wherein the inflammatory condition is selected from the inflammatory condition is selected from Systemic Sclerosis-Associated Interstitial Lung Disease (SSc-ILD); septic shock; chronic ABMR (antibody-mediated rejection); Adult Onset Still Disease (AOSD); Neuromyelitis optica spectrum disorder (NMOSD); and Multicentric Castleman's disease (MCD).

18. The method of claim 17, wherein the inflammatory condition is Multicentric Castleman's disease (MCD) and the patient is human immunodeficiency virus (HIV) negative and human herpesvirus-8 (HHV-8) negative.

19. The method of any one of claims 10-18, wherein the mavorixafor or a pharmaceutically acceptable salt thereof is administered orally in an amount from about 200 mg to about 1200 mg daily.

20. The method of claim 9, wherein the amount of mavorixafor or a pharmaceutically acceptable salt thereof is about 200 mg to about 600 mg daily.

21. The method of claim 19 or claim 20, wherein the mavorixafor or a pharmaceutically acceptable salt thereof is administered twice a day in divided dosage, approximately 12 hours apart.

22. The method of claim 19 or claim 20, wherein the mavorixafor or a pharmaceutically acceptable salt thereof is administered once daily.

23. The method of any one of claims 10-22, wherein the dose of the IL-6 inhibitor required for effective treatment is decreased by at least 20% relative to the effective dose of the IL-6 inhibitor as a monotherapy.
